SEQUENCE LISTING (6 sequences)

SEQ ID NO: 1 - DNA, 1503 bp, synthetic construct
  source 1..1503: mol_type = other DNA
gctcaggacgaacgctggcggcgtgcctaatacatgcaagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagtttgaaagatggcttcggctatcacttttggatggtcccgcggcgtattagctagatggtgaggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcctaaggtgggacagatgattagggtgaagtcgta

SEQ ID NO: 2 - DNA, 710 bp, synthetic construct
  source 1..710: mol_type = other DNA
atttcggtaaaggggcgattatgcggatgggtgacgctgcccagacgactatttcaacgatttcaagtggatcgttggccttagatgacgcgttgggtgtcggcggctacccacgtggtcggatcgtggaaatctacggtcctgaaagttcaggtaaaacgaccgtggcactacatgcggttgctgaagttcaaaagcagggtggtacggcggcctatatcgatgctgaaaacgcactagaccccgtttatgcggaacacctaggggtcaacattgatgacctgttactttcgcaaccagatactggtgaacaagggcttgaaattgcagatgccttagtttccagtggtgcggtcgatattttagttgttgactcggtggcggccttagtgccacgtgccgaaattgaaggtgaaatgggtgacgcacacgttgggttacaagcgcggctgatgtcacaagcgctccggaagttatcagggacattgaacaaaaccaagacaatcgcgttatttatcaatcaaattcgtgaaaaagttggtgtgatgtttggtaatcctgaaacgactcctggtggtcgggccttgaaattttacgccacgattcgtttggaagtacggcgggcagaacagatcaaggaaggaaccaatattattggtaaccgtgtccggatcaaagtcgttaagaacaaggttgcaccccattttaag

SEQ ID NO: 3 - DNA, 20 bp, synthetic construct
  source 1..20: mol_type = other DNA
agagtttgatcmtggctcag

SEQ ID NO: 4 - DNA, 22 bp, synthetic construct
  source 1..22: mol_type = other DNA
tacggytaccttgttacgactt

SEQ ID NO: 5 - DNA, 19 bp, synthetic construct
  source 1..19: mol_type = other DNA
ctgatgcacggaaagcagc

SEQ ID NO: 6 - DNA, 21 bp, synthetic construct
  source 1..21: mol_type = other DNA
cgcttaaatggyggygcmacc

(19) United States
(12) Patent Application Publication
(10) Pub. No.: US 2026/0216262 A1
(43) Pub. Date: Jul. 30, 2026

(54) USE OF LACTIPLANTIBACILLUS PLANTARUM STRAIN FOR IMPROVING SKIN CONDITIONS CAUSED BY ALLERGIES

(71) Applicants: GI BIOME INC, Seongnam-si (KR); MAEIL DAIRIES CO., LTD., Seoul (KR)

(72) Inventors: Bo Gie YANG, Seoul (KR); Myung Ho JANG, Seoul (KR); A-Ram KIM, Namyangju-si (KR); Seok Jun PARK, Seoul (KR); Jin Ah YOO, Osan-si (KR); Chung Hoo YOON, Yongin-si (KR); Byung Min LEE, Hwaseong-si (KR); Yong Won YOON, Osan-si (KR)

(21) Appl. No.: 19/142,169

(22) PCT Filed: Dec. 22, 2023
(86) PCT No.: PCT/KR2023/021419

§ 371 Date: Jun. 20, 2025

(30) Foreign Application Priority Data
Dec. 22, 2022 (KR) 10-2022-0182250
Jun. 29, 2023 (KR) 10-2023-0083976

(51) Int. Cl. A61K 35/747; A61P 17/00; A61P 37/08

(52) U.S. Cl. CPC A61K 35/747; A61P 17/00; A61P 37/08

(57) ABSTRACT

The present invention relates to use of a Lactiplantibacillus plantarum strain for ameliorating skin conditions caused by allergies. The strain according to an aspect, and a lysate derived from the strain, a culture derived from the strain, an extract of the culture, or a mixture thereof may be effective for the prevention, amelioration, or treatment of allergic disease.

### TECHNICAL FIELD

The present invention relates to use of a strain of Lactiplantibacillus plantarum for ameliorating a skin condition caused by allergies.

### BACKGROUND ART

Allergy or allergic disease is a compound word created by combining the Greek words ‘allos’ meaning ‘to change’ and ‘ergo’ meaning ‘to act’, and thus is defined as an adverse biological reaction. As the etymology suggests, an allergy is a congenital or acquired abnormality of the immune system that causes a hypersensitivity reaction in some people, such as hives, itching, runny nose, or coughing, to an allergen which is a substance causing no reaction in others. Typical allergens include pollen, drugs, plant fibers, bacteria, molds, house dust mites, foods (egg albumin, milk proteins, peanuts), dyes, chemicals, and the like, and in recent years, allergic diseases have been increasing worldwide due to increased indoor living, an increase in allergens such as new chemicals, environmental pollution, decreased immune function due to stress, and dietary changes. Representative allergic diseases include atopic dermatitis, bronchial asthma, allergic rhinitis, allergic keratitis, skin urticaria, and the like, and mast cells are known as important mediating cells in these diseases.

Allergic reactions are induced by activation of intracellular signaling pathways when allergens bind to IgE bound to the high-affinity IgE receptor (FcεRI) on the surface of the mast cells. Specifically, when allergens invade the human body, T-helper cells and B cells are sequentially activated to produce IgE. The allergens that invade the human body are then recognized by antigen-presenting cells, and the antigen-presenting cells differentiate T helper cell type 0 (Th0) into Th2 cells through allergen presentation. These differentiated Th2 cells secrete cytokines, such as IL-4, IL-5, and IL-13, which not only promote development of eosinophils in the bone marrow and drive eosinophils to inflamed tissues, but also act on B cells to induce the production of IgE. The resulting IgE binds to the high-affinity IgE receptor (FcRIα) on the mast cell membrane, and when allergens such as mites or pollen bind thereto and form a cross-link, the receptor (FcRIα) aggregates. By cross-linking and aggregation of the receptor (FcRIα), the mast cells are activated, resulting in an increase in the concentration of calcium ions (Ca++) in the cytoplasm. The increase in the concentration of calcium ions acts on various types of intracellular regulators, such as cytoskeleton and cholesterol in the cell membrane, to move granules to the cell membrane, and at the same time, causes degranulation through vacuolization of the granules, mutual fusion of the granules, fusion of the granules with the cell membrane, and the like. When degranulation occurs, histamine, prostaglandins, leukotrienes, serotonin, and the like are secreted, causing vasodilation, increased vascular permeability, contraction of bronchial smooth muscles, and the like, and degranulation of mast cells is accompanied by secretion of inflammatory cytokines such as IL-1β, IL-4, IL-5, IL-6, TNF-α, and the like so that neutrophils, eosinophils, macrophages, Th2 cells, basophils, and the like infiltrate the relevant tissue to trigger an allergic inflammatory response.

Therefore, substances that inhibit the activation of mast cells, inhibit the degranulation of mast cells, or inhibit production of inflammatory cytokines may be effective therapeutic drugs for allergies. Currently available anti-allergic drugs include antihistamines (alkylamines, desloratadine, etc.), mast cell stabilizers (cromoglycate, etc.), leukotriene blockers (montelukast, etc.), anti-IgE antibodies (omalizumab), and the like. However, effects of these drugs are often temporary, and continuous treatment at strong concentrations includes side effects, and thus these drugs are often limited in their use in treating allergic diseases which are chronic immune diseases.

Therefore, there is still a need to develop new substances that can prevent and ameliorate allergic disease, with fewer side effects and lasting effects.

### DISCLOSURE OF INVENTION

### Technical Problem

One aspect is to provide a Lactiplantibacillus plantarum strain having anti-allergic activity, wherein the anti-allergic activity is reducing an IgE concentration in serum or inhibiting degranulation activity of mast cells.

Another aspect is to provide a Lactiplantibacillus plantarum HD-02 strain deposited with Accession No. KACC81228BP and belonging to the Lactiplantibacillus sp.

Another aspect is to provide a lysate derived from the strain, a culture derived from the strain, or an extract of the culture.

Another aspect is to provide a food composition including, as an active ingredient, a Lactiplantibacillus plantarum strain, a lysate derived from the strain, a culture derived from the strain, or a mixture thereof.

Another aspect is to provide a health functional food for preventing or ameliorating allergic disease, the health functional food including, as an active ingredient, a Lactiplantibacillus plantarum strain, a lysate derived from the strain, a culture derived from the strain, or a mixture thereof.

Another aspect is to provide a pharmaceutical composition for treating or preventing allergic disease, the pharmaceutical composition including, as an active ingredient, a Lactiplantibacillus plantarum strain, a lysate derived from the strain, a culture derived from the strain, or a mixture thereof.

Another aspect is to provide a cosmetic composition for preventing or ameliorating allergic disease, the cosmetic composition including, as an active ingredient, a Lactiplantibacillus plantarum strain, a lysate derived from the strain, a culture derived from the strain, or a mixture thereof.

Another aspect is to provide a feed composition for preventing or ameliorating allergic disease, the feed composition including, as an active ingredient, a Lactiplantibacillus plantarum strain, a lysate derived from the strain, a culture derived from the strain, or a mixture thereof.

Another aspect is to provide a method of preventing or treating allergic disease, the method including administering an effective amount of a composition to a subject in need thereof, the composition including a Lactiplantibacillus plantarum HD-02 strain deposited with Accession No. KACC81228BP, a culture of the strain, a lysate of the strain, or a mixture thereof.

Another aspect is to provide use of a composition for preparation of a pharmaceutical agent or health functional food agent for preventing or treating allergic disease, the composition including a Lactiplantibacillus plantarum HD-02 strain deposited with Accession No. KACC81228BP, a culture of the strain, a lysate of the strain, or a mixture thereof.

### Solution to Problem

One aspect provides a Lactiplantibacillus plantarum strain deposited with Accession No. KACC81228BP and belonging to the Lactiplantibacillus sp.

Lactiplantibacillus is a gram-positive aerobic or facultative anaerobic microorganism of the genus Bacillus that is widely distributed in nature. Microorganisms belonging to the genus Lactobacillus may include Lactiplantibacillus plantarum, Lactiplantibacillus fermentum, and the like. The inventors of the present invention conducted research to develop a new strain having excellent anti-allergic effects, and selected Lactiplantibacillus plantarum HD-02 as a candidate anti-allergic strain. This strain was deposited with Accession No. KACC81228BP at the Korean Agricultural Culture Collection on Aug. 26, 2022. This strain is a probiotic strain, is harmless to the human body, and can be used without a side effect.

In an embodiment, the Lactiplantibacillus plantarum strain may be a strain deposited with Accession No. KACC81228BP.

In an embodiment, the Lactiplantibacillus plantarum strain may be a strain having 16S rRNA of SEQ ID NO: 1.

In an embodiment, the strain may be a strain having 16S rRNA consisting of a nucleotide sequence of SEQ ID NO: 1 or 16S rRNA including a nucleotide sequence having at least 97% nucleotide sequence identity with the nucleotide sequence of SEQ ID NO: 1. Specifically, the nucleotide sequence identity with the nucleotide sequence consisting of SEQ ID NO: 1 of the present specification may be at least 93%, 95%, 96%, 97%, 98%, 99%, 99.5%, 99.8%, 99.9%, or 100%.

In an embodiment, the Lactiplantibacillus plantarum strain may be a strain having the recA gene sequence of SEQ ID NO: 2.

In an embodiment, the strain may be a strain having the recA gene sequence of SEQ ID NO: 2 or recA having at least 97% gene sequence identity with the recA gene sequence of SEQ ID NO: 2. Specifically, the gene sequence identity with the gene sequence consisting of SEQ ID NO: 2 of the present specification may be at least 94%, 95%, 96%, 97%, 98%, 99%, 99.5%, 99.8%, 99.9%, or 100%.

In an embodiment, the strain may be a naturally occurring mutant strain.

In an embodiment, the strain may be a live bacterium, a dead bacterium, or a cytoplasmic fraction obtained by disrupting the strain, and preferably, may preferably be a live bacterium. The strain may exist as a live microbial cell or a dead microbial cell, or in a dried state or a lyophilized state.

In an embodiment, the strain may have anti-allergic activity (hereinafter, the terms allergy and allergic disease may be used interchangeably).

In an embodiment, the strain may have activity of inhibiting production of Th2-mediated cytokines, inhibiting degranulation of mast cells, inhibiting IgE levels in serum, or inhibiting MCPT-1 levels in serum. In detail, the activity of inhibiting degranulation of mast cells may be inhibiting secretion of β-hexosaminidase from activated mast cells.

In an embodiment, the strain may reduce the IgE concentration in serum or reduce binding of IgE to a surface receptor of the mast cells. In addition, the strain may inhibit MCPT-1 levels in serum.

Therefore, the strain according to an embodiment may be useful in the treatment of allergic disease.

Another aspect provides a lysate derived from the Lactiplantibacillus plantarum strain, a culture derived from the strain, an extract of the culture, or a mixture thereof.

In the present specification, the term “culture” may be used interchangeably with “culture product,” “culture supernatant,” “supernatant liquid of a culture product,” “conditioned culture,” or “conditioned medium,” and may refer to the entire medium containing a strain, a metabolite of the strain, extra nutrients, and the like, the strain being obtained by culturing the Lactiplantibacillus sp. strain for a certain period of time in a medium capable of supplying nutrients for the strain to grow and survive in vitro. The culture product may refer to a product obtained by culturing a probiotic strain in a known medium, and this product may or may not include the strain itself. The medium may be selected from known liquid media or known solid media. For example, the medium may be an MRS liquid medium, a GAM liquid medium, an MRS agar medium, a GAM agar medium, a BL agar medium, or the like, but is not limited thereto.

The culture may contain a culture itself obtained by culturing the strain, a concentrate of the culture, or a lyophilized product of the culture, or may contain a culture supernatant obtained by removing the strain from the culture, a concentrate of the culture supernatant, or a lyophilized product of the culture supernatant.

In the present specification, the term “lysate” may be used interchangeably with “dissolved product”, and may refer to a solution or suspension of an aqueous medium of cells of a microorganism such as broken Lactiplantibacillus plantarum. A cell lysate contains, for example, macromolecules such as DNA, RNA, proteins, peptides, carbohydrates, lipids, etc., and/or small molecules such as amino acids, sugars, fatty acids, etc., or fractions of the macromolecules or small molecules. In addition, the dissolved product contains cell debris, which may be smooth or granular in structure.

In the present specification, the term “extract of the culture” refers to a product obtained by extracting the culture or a concentrate thereof, and may include an extracted solution, a diluted or concentrated solution of the extracted solution, a dried product obtained by drying the extracted solution, or a crude purified product or purified product of the extracted solution, or a fraction obtained by fractionating the extracted solution.

The culture of the present invention may include a live bacteria culture (i.e., a resulting product obtained by culturing live bacteria in a medium), a culture obtained by killing live cells in the live bacteria culture, or a culture filtrate (i.e., a resulting product obtained by removing dead microbial cells or live microbial cells from the culture). In addition, components derived from the strain of the present application may include, for example, a cell wall structure obtained by disrupting a cell body, or a cytoplasmic fraction containing extracellular vesicles (EVs). In addition, a form of lactic acid bacteria suitable for inclusion in the pharmaceutical composition, a formulation method, and a method of isolating components derived from the strain are well known to a person skilled in the art.

In an embodiment, the culture may be obtained by culturing Lactiplantibacillus plantarum in an appropriate medium (e.g., an MRS plate medium) at a temperature of higher than 10° C. or lower than 40° C., for a certain period of time, for example, 4 to 50 hours.

In an embodiment, a culture medium and culture conditions for culturing the Lactiplantibacillus plantarum may be appropriately selected or modified for use by a person skilled in the art.

Another aspect provides a composition including a lysate derived from the Lactiplantibacillus plantarum strain, a culture of the strain, an extract of the culture, or a mixture thereof.

In an embodiment, the composition may further include an additive selected from the group consisting of a preservative, a dye, an emulsifier, a sweetener, a stabilizers, a flavor enhancer, a flavoring agent, and an acidifier.

In an embodiment, a dosage form of the composition may be at least one selected from the group consisting of a solution, an emulsion, a viscous mixture, a powder, a granule, a tablet, and a capsule.

In an embodiment, the composition may have anti-allergic activity.

In an embodiment, the anti-allergic activity may be inhibiting production of helper T cell type 2 (Th2)-mediated cytokines or alleviating an allergic reaction, by including the strain as an effective ingredient. This activity may include alleviating an immune hypersensitivity reaction, specifically, ameliorating or alleviating skin conditions, such as atopic dermatitis, caused by the immune hypersensitivity reaction.

In an embodiment, the anti-allergic activity may include inhibiting degranulation in activated mast cells. Specifically, the anti-allergic activity may include inhibiting secretion of β-hexosaminidase, histamine, prostaglandins, and serine proteases from activated mast cells.

In an embodiment, the anti-allergic activity may include reducing the IgE concentration in serum or reducing binding of IgE to a surface receptor of mast cells. In addition, the anti-allergic activity may include inhibiting MCPT-1 levels in serum.

In an embodiment, the anti-allergic activity may include activity of inhibiting degranulation of mast cells by 50% to 70% when the Lactiplantibacillus plantarum HD-02 strain is treated with RBL-2H3 mast cells. In an embodiment, the anti-allergic activity may include activity of inhibiting an allergic reaction by 20% to 40% when the Lactiplantibacillus plantarum HD-02 strain is treated in an animal model with passive cutaneous anaphylaxis (PCA) induced by anti-DNP IgE.

According to an embodiment, the anti-allergic activity may include activity of inhibiting ear edema by 40% to 60%, activity of reducing the IgE concentration in serum by 50% to 70%, or activity of reducing the MCPT-1 concentration in serum by 50% to 70%, when the Lactiplantibacillus plantarum HD-02 strain is treated in an animal model with MC903-induced atopic dermatitis.

In an embodiment, the anti-allergic activity may include activity of reducing the IgE concentration in serum by 50% to 70%, activity of reducing transepidermal moisture loss by 30% to 60%, activity of alleviating itching by 40% to 70%, activity of reducing the number of activated immune cells (e.g., B cells, T cells, Th1 cells, and Th2 cells) in lymph nodes (e.g., axillary lymph nodes) caused by an immune hypersensitivity reaction by 50% to 90%, activity of inhibiting edema by 40% to 60%, or activity of reducing the number of activated mast cells in inflamed tissue by 50% to 70%, when the Lactiplantibacillus plantarum HD-02 strain is treated in an animal model with house dust mite-induced atopic dermatitis (a severe atopic dermatitis model).

In an embodiment, the Lactiplantibacillus plantarum HD-02 strain may have activity of alleviating allergic reaction symptoms or atopic dermatitis symptoms by 90% or less, 80% or less, 70% or less, 67% or less, 10 to 90%, 10 to 80%, 10 to 70%, 10 to 67%, 20 to 90%, 20 to 80%, 20 to 70%, 20 to 67%, 30 to 90%, 30 to 80%, 30 to 70%, 30 to 67%, 40 to 90%, 40 to 80%, 40 to 70%, 40 to 67%, 50 to 90%, 50 to 80%, 50 to 70%, 50 to 67%, or 60 to 67%, based on 100% of allergy reaction symptoms in a control group in which an allergic reaction or atopic dermatitis is induced without administering the strain.

Another aspect provides use of a Lactiplantibacillus plantarum strain, a lysate of the strain, a culture of the strain, or an extract of the culture for amelioration, prevention, or treatment of disease.

In an embodiment, the use may include prevention, amelioration, or treatment of immunoglobulin E (IgE)-mediated disease or condition. Specifically, the IgE-mediated disease may be an allergic disease.

In the present specification, “allergic disease” refers to hypersensitivity to a specific substance in the human body, i.e., a condition, disease, or abnormality caused by an excessive response of the immune system to a substance introduced from outside the body, and the substance introduced from outside may be an allergen which is an antigen that causes an allergic disease. The allergy may refer to hypersensitivity reaction in which an inflammatory mediator, such as histamine, is dissociated by a substance introduced from outside and causes a disease, and the hypersensitivity reaction may be a type 1 hypersensitivity reaction, a type 2 hypersensitivity reaction, a type 3 hypersensitivity reaction, or a type 4 hypersensitivity reaction.

In an embodiment, the allergic disease may include an allergic reaction caused by house dust, fungi, mites, or hair, skin or excrement of an animal.

Non-limiting examples of the allergic disease may include at least one disease selected from the group consisting of edema, anaphylaxis, rhinitis, allergic rhinitis, asthma, allergic conjunctivitis, allergic dermatitis, allergic tympanitis, atopic dermatitis, contact dermatitis, urticaria, chronic spontaneous urticaria, hives, dry eye, pruritus, anaphylactic shock, pollen allergy, bacterial allergy, fungal allergy, viral allergy, insect allergy, food allergy, drug allergy, and respiratory allergy, but are not limited thereto.

In the present specification, the term “atopic dermatitis” refers to a chronic and relapsing eczematous skin disease causing severe itching, and in chronic cases, the epidermis is thickened and infiltrated with cells involved in various immune responses. The skin of a patient with atopic dermatitis appears dry and lusterless. The skin with dryness is more prone to moisture loss due to a weakened skin barrier function, and external irritants can easily penetrate the skin.

Atopic dermatitis is characterized by red spots with severe itching, small convex papules, blisters, scratches, and oozing during the acute phase, which can be accompanied by secondary infections. As the lesion progresses, the subacute phase may result in scratched wounds and scaly red papules. In the chronic phase, although not limited thereto, repeated scratching of the skin may result in lichenification, which is a condition in which the skin becomes rough and thick and wrinkles become apparent, and red or brownish nodules accompanied by severe itching (Prurigo Nodularis) may occur.

In the present specification, the term “urticaria” is defined as a disease characterized by symptoms of an itchy rash and/or angioedema, and the term “chronic urticaria” is defined as urticaria that exists continuously or intermittently for at least 6 weeks.

In the present specification, the term “asthma” refers to a disease in which the bronchi in the lungs become very sensitive, sometimes narrowing the bronchi, resulting in shortness of breath, wheezing, and severe coughing. The asthma is an allergic disease caused by an allergic inflammatory reaction in the bronchi, and includes bronchial asthma, allergic asthma, and non-allergic asthma.

Allergic rhinitis and bronchial asthma are representative respiratory allergic diseases, wherein the allergic rhinitis is an inflammatory disease of the upper respiratory tract, and asthma is an inflammatory disease of the lower respiratory tract. Both diseases are IgE-mediated inflammatory diseases characterized by airway hyperresponsiveness and increased reactivity to irritants. At the same time, eosinophils, mast cells, and Th2 cells are shared as common inflammatory cells and common inflammatory factors such as histamine, leukotrienes, and Th2 cytokines may be produced. Accordingly, it is suggested that the two diseases are closely related.

Allergic rhinitis may be one of the most common chronic inflammatory diseases in both adults and children. The nasal mucosa is hypersensitive to a variety of causative substances, with the main symptoms being a clear runny nose, a congested nose, an itchy nose, and sneezing, and often accompanied by eye symptoms such as itchy eyes, red eyes, and the like. The most common causes of allergic rhinitis include pollen, dust mites, and pet hair, and symptoms may be exacerbated by air pollution and rapid temperature changes. Well-known aggravating factors include exercise, allergens or irritants, weather changes, and respiratory viral infections including cold. Since several epidemiologic and pathophysiologic studies have confirmed the close relationship between the two diseases, the concept of “one airway disease” or “united disease” has been proposed, in which the two allergic inflammatory diseases of the upper and lower airways are considered as a single airway disease rather than as separate diseases. Therefore, it can be seen that a comprehensive approach is recommended in a way that patients with rhinitis should be checked for comorbid asthma and patient with asthma should be also checked for the presence of rhinitis as a comorbidity at the same time.

In the present specification, the term “house dust mites” refers to a case where carcass or feces irritate the skin mucosa so that histamine, an inflammatory substance in the human body, is released and exacerbate atopic dermatitis.

The composition of the present invention may induce normalization of the intestinal microbiota modified by allergic diseases, thereby exhibiting excellent effects in the control, prevention, amelioration, and treatment of an allergic disease and complications thereof, in addition to the aforementioned effects in the control, prevention, amelioration, and treatment of an allergic disease.

In the present specification, the term “treat” refers to any act of ameliorating or beneficially altering symptoms of an allergic disease by administration of the pharmaceutical composition of the present invention.

In the present specification, the term “prevent” refers to any act of inhibiting symptoms of an allergic disease or delaying progression of an allergic disease by administration of the pharmaceutical composition of the present invention.

In the present specification, the wording “including as an active ingredient” refers to addition of a strain of the present specification, a lysate of the strain, a culture of the strain, or an extract of the culture to an extent capable of exhibiting the aforementioned effects, and includes formulations in which various ingredients are added as auxiliary ingredients for drug delivery and stabilization.

In an embodiment, the composition may include 0.001 wt % to 80 wt % of the Lactiplantibacillus plantarum strain based on the total weight of the composition. In addition, a dosage of the Lactiplantibacillus plantarum strain may be 0.01 mg to 10,000 mg, 0.1 mg to 1,000 mg, 1 mg to 100 mg, 0.01 mg to 1,000 mg, 0.01 mg to 100 mg, 0.01 mg to 10 mg, or 0.01 mg to 1 mg. The composition may include the strain in a therapeutically effective amount or in a nutritionally effective concentration. For example, the composition may include the strain in an amount of 10³ to 10¹⁶ CFU/g, 10³ to 10¹⁵ CFU/g, 10³ to 10¹⁴ CFU/g, 10³ to 10¹³ CFU/g, 10³ to 10¹² CFU/g, 10⁴ to 10¹⁶ CFU/g, 10⁴ to 10¹⁵ CFU/g, 10⁴ to 10¹⁴ CFU/g, 10⁴ to 10¹³ CFU/g, 10⁴ to 10¹² CFU/g, 10⁵ to 10¹⁶ CFU/g, 10⁵ to 10¹⁵ CFU/g, 10⁵ to 10¹⁴ CFU/g, 10⁵ to 10¹³ CFU/g, 10⁵ to 10¹² CFU/g, 10⁶ to 10¹³ CFU/g, 10⁶ to 10¹² CFU/g, 10⁷ to 10¹³ CFU/g, 10⁷ to 10¹² CFU/g, 10⁸ to 10¹³ CFU/g, or 10⁸ to 10¹² CFU/g, or may include cultures of an equivalent number of live bacteria or dead bacteria. Specifically, in the case of adult patients, 1×10³ to 1×10¹⁶ CFU/g of live bacteria or dead bacteria may be divided into several portions and administered multiple times. However, the dosage may be prescribed in various ways depending on factors such as a formulation method, an administration method, a patient's age, weight, and gender, pathological conditions, food, administration time, an administration route, an excretion rate, and reaction sensitivity, and a person skilled in the art may appropriately adjust the dosage in consideration of these factors. The number of administrations may be once or at least twice within the range of clinically acceptable side effects, and the number of administration sites may be one or at least two. For non-human animals, the same dosage as that for humans per kg (of body weight) may be administered, or for example, the dosage is calculated based on the volume ratio (e.g., average value) of organs (e.g., the heart) between a target animal and humans and then administered. Possible routes of administration may include oral, sublingual, parenteral (e.g., subcutaneous, intramuscular, intraarterial, intraperitoneal, intrathecal, or intravenous), rectal, topical (including transdermal), and inhale injection, implantable devices, or insertion of substances. Examples of animals to be treated according to an embodiment include humans and other target mammals, and specifically include humans, monkeys, mice, rats, rabbits, sheep, cows, dogs, horses, pigs, and the like. According to an embodiment, the composition includes a killed dry strain, and may be administered in the amount of 1 g to 10 g, 0.5 g to 1.5 g, 2.5 g to 3.5 g, or 4.5 g to 5.5 g at a time, once a day to three times a day.

Another aspect provides a food composition including, as an active ingredient, a Lactiplantibacillus plantarum strain, a lysate derived from the strain, a culture derived from the strain, or a mixture thereof.

Another aspect provides a health functional food for preventing or ameliorating allergic diseases, including, as an active ingredient, a Lactiplantibacillus plantarum strain, a lysate derived from the strain, a culture derived from the strain, or a mixture thereof.

In an embodiment, the food composition or the health functional food may have anti-allergic activity.

The terms “strain”, “anti-allergic activity,” and “allergic disease” are as defined above.

In an embodiment, the ameliorating an allergic disease may include alleviating an immune hypersensitivity reaction or ameliorating a skin condition caused by an immune hypersensitivity reaction.

In an embodiment, the ameliorating an allergic disease may include inhibiting Th2-mediated cytokine production, inhibiting degranulation activity of mast cells, or inhibiting levels of IgE or MCPT-1 in serum.

In an embodiment, the allergic disease may include an allergic reaction caused by house dust, fungi, mites, or hair, skin or excrement of an animal.

The allergic disease may be selected from the group consisting of edema, anaphylaxis, rhinitis, allergic rhinitis, asthma, allergic conjunctivitis, allergic dermatitis, allergic tympanitis, atopic dermatitis, contact dermatitis, urticaria, chronic spontaneous urticaria, hives, dry eye, pruritus, anaphylactic shock, pollen allergy, bacterial allergy, fungal allergy, viral allergy, insect allergy, food allergy, drug allergy, and respiratory allergy.

In an embodiment, the food composition may be for preventing or ameliorating an allergic disease.

In the food composition, the food includes meat, sausage, bread, chocolates, candies, snacks, confectionery, cereals, bakery products, dairy products, pizza, ramen, other noodles, gums, dairy products including ice cream, various types of powdered milk, infant formula, milk, milk powder, yogurt, cheese, fermented milk, soup, beverages, tea, drinks, alcoholic beverages, vitamin supplements, food additives, health functional food, and health food, and includes all foods in the conventional sense.

The health functional food is the same as food for special health use (FoSHU), which means food with high medical and healthcare effects that has been processed to efficiently exhibit bioregulatory functions in addition to providing nutrition. Here, “functional” means to have a useful effect on the structure and function of the human body for health purposes, such as regulating nutrients or physiological actions. The food of the present application may be prepared by methods commonly used in the art, and may be prepared by adding raw materials and ingredients commonly added in the art. In addition, regarding formulation of the food, the food may be prepared in any formulation recognized as a food. The food composition of the present application may be prepared in various types of formulations, and since it uses food as a raw material unlike general pharmaceuticals, the food composition has advantages of being free from side effects that may occur with long-term use of pharmaceuticals and is highly portable. In this regard, the HD-02 of the present application may be used for the preparation of a supplement to enhance the effect of preventing or ameliorating allergies.

The health food refers to food that has a more active health maintenance or promotion effect than general food, and the health supplement food refers to food for the purpose of health supplementation. In some cases, the terms health functional food, health food, and health supplement may be used interchangeably.

Specifically, the health functional food refers to a food product prepared by adding the composition according to an embodiment to a food material, such as a beverage, tea, spice, chewing gum, or confectionery, or may be prepared by encapsulation, pulverization, suspending. The health functional food is meant to exhibit a specific health effect when consumed, but since it uses food as a raw material unlike general pharmaceuticals, the health functional food has advantages of being free from side effects that may occur with long-term use of pharmaceuticals.

The food composition according to an embodiment may be very useful because it can be consumed on a daily basis and thus can be expected to be highly effective in preventing or ameliorating allergic diseases.

The food composition may further include a physiologically acceptable carrier. The type of carrier is not particularly limited, and any carrier commonly used in the art may be used.

In addition, the food composition may include additional ingredients commonly used in food compositions to improve odor, taste, appearance, and the like. For example, vitamins A, C, D, E, B1, B2, B6, and B12, niacin, biotin, folate, pantothenic acid, and the like may be included. In addition, minerals such as zinc (Zn), iron (Fe), calcium (Ca), chromium (Cr), magnesium (Mg), manganese (Mn), copper (Cu), chromium (Cr), and the like may be included. In addition, amino acids such as lysine, tryptophan, cysteine, valine, and the like may be included.

In addition, the food composition may include food additives such as preservatives (potassium sorbate, sodium benzoate, salicylic acid, sodium dehydroacetate, etc.), bactericides (bleaching powder and highly bleaching powder, sodium hypochlorite, etc.), antioxidants (butylated hydroxyanisole (BHA), butylated hydroxytoluene (BHT), etc.), colorants (tar color, etc.), color developers (sodium nitrite, etc.), bleaching agents (sodium sulfite), seasonings (monosodium glutamate (MSG), etc.), sweeteners (dulcin, cyclamate, saccharin, sodium, etc.), flavorings (vanillin, lactones, etc.), swelling agents (alum, D-potassium hydrogen tartrate, etc.), enhancers, emulsifiers, thickeners (gelling agents), film agents, gum bases, foam suppressants, solvents, improvers, and the like. The additives may be selected and used in appropriate amounts depending on the type of food.

The composition according to an embodiment may be added as it is or used together with other foods or food ingredients, and may be used appropriately according to conventional methods. A mixing amount of the active ingredient may be appropriately determined depending on the purpose of its use (e.g., prevention, health, or therapeutic treatment). In general, when preparing food or beverages, the food composition of the present application may be added in an amount of 50 parts by weight or less, specifically 20 parts by weight or less. However, in the case of long-term intake for the purpose of health and hygiene, the amount may be less than these ranges, and since there is no safety issue, the active ingredient may be used in the amount greater than these ranges.

As an example of the food composition, the health beverage composition may be used, and in this case, various flavoring agents or natural carbohydrates may be contained as additional ingredients like a regular beverage. The natural carbohydrates may be: monosaccharides such as glucose and fructose; disaccharides such as maltose and sucrose; polysaccharides such as dextrin and cyclodextrin; and sugar alcohols such as xylitol, sorbitol and erythritol. The sweeteners may include: natural sweeteners such as thaumatin and a stevia extract; and synthetic sweeteners such as saccharin and aspartame. A proportion of the natural carbohydrates may be generally, per 100 ml of the health beverage composition of the present application, about 0.01 to 0.04 g, specifically about 0.02 to 0.03 g per.

In addition to these ingredients, the health beverage composition may contain various nutrients, vitamins, electrolytes, flavors, colorants, pectic acid and salts thereof, alginic acid and salts thereof, organic acids, protective colloid thickeners, pH adjusters, stabilizers, antiseptics, glycerin, alcohols, or carbonating agents. In addition, fruit pulp for the preparation of natural fruit juice, fruit juice beverage, or vegetable beverage may be contained. Such components may be used independently or in combination. A proportion of these additives is not so important, but is generally selected in a range of 0.01 to 0.1 parts by weight based on 100 parts by weight of the health beverage composition of the present application.

The food composition according to an embodiment may be included in various wt % if it can exhibit an effect of preventing or ameliorating allergic diseases. For example, the HD-02 according to an embodiment may be included, based on the total weight of the food composition, in an amount of 0.0001 to 100 wt %, 0.0001 to 80 wt %, 0.0001 to 50 wt %, 0.0001 to 40 wt %, 0.0001 to 20 wt %, 0.0001 to 10 wt %, 0.0001 to 5 wt %, 0.0001 to 2 wt %, 0.0001 to 1 wt %, 0.0001 to 0.5 wt %, 0.0001 to 0.2 wt %, 0.0001 to 0.1 wt %, 0.0001 to 0.05 wt %, 0.0001 to 0.02 wt %, 0.0001 to 0.01 wt %, 0.0001 to 0.005 wt %, 0.0001 to 0.002 wt %, or 0.0001 to 0.001 wt %, but is not limited thereto, and may be included by conventional wt % in the art without limitation. Specifically, the fermented milk may include the HD-02 in an amount of, based on the total weight of the food composition, 0.0001% to 0.01 wt %, 0.0001% to 0.005 wt %, 0.0001% to 0.003%, 0.0001% to 0.002 wt %, or 0.0001% to 0.001 wt %. Specifically, the beverage may include the HD-02 in an amount of, based on the total weight of the food composition, 0.0001% to 0.2 wt %, 0.0001% to 0.1 wt %, 0.0001% to 0.05 wt %, 0.0001% to 0.02 wt %, or 0.0001% to 0.01 wt %. Specifically, the powdered or solid food may include the HD-02 in an amount of, based on the total weight of the food composition, 0.001% to 50 wt %, 0.001% to 30 wt %, 0.001% to 25 wt %, 0.001% to 20 wt %, or 0.001% to 10 wt %. The mixing amount of the active ingredient may be suitably determined depending on the purpose of its use (for prevention, health, or symptom relief). For example, the Lactiplantibacillus plantarum HD-02 may be included at a concentration of 10³ CFU/g to 10¹⁶ CFU/g, but is not limited thereto.

In the health functional food, the strain or culture thereof may be used alone or in combination with other foods or food ingredients, and may be used suitably used according to conventional methods. The mixing amount of the active ingredients may be suitably determined depending on the purpose of its use (for prevention, health, or therapeutic treatment). Generally, in the preparation of a food or beverage, the composition of the present specification may be added in an amount of 15 parts by weight or less with respect to raw materials. Types of the health functional food are not particularly limited. From among types of the health functional food, the beverage composition may include additional ingredients, such as various flavorings or natural carbohydrates, as in a regular beverage. The natural carbohydrates may be monosaccharides, such as glucose and fructose, disaccharides, such as maltose and sucrose, polysaccharides, such as dextrin and cyclodextrin, and sugar alcohols, such as xylitol, sorbitol and erythritol. The sweeteners may include natural sweeteners, such as thaumatin and a stevia extract, and synthetic sweeteners, such as saccharin and aspartame. The health food composition may also include a nutrient, a vitamin, an electrolyte, a flavor, a colorant, pectic acid and a salt thereof, alginic acid and a salt thereof, organic acid, a protective colloid thickener, a pH adjuster, a stabilizer, an antiseptic, glycerin, alcohol, a carbonating agent, or a combination thereof. The health functional food composition may also include fruit pulp for the preparation of natural fruit juice, fruit juice beverage, or vegetable beverage, or a combination thereof.

Another aspect provides a pharmaceutical composition for treating or preventing allergic diseases, the pharmaceutical composition including, as an active ingredient, a Lactiplantibacillus plantarum strain, a lysate derived from the strain, a culture derived from the strain, or a mixture thereof.

The terms “strain”, “anti-allergic activity,” and “allergic disease” are as defined above.

In an embodiment, the pharmaceutical composition may have anti-allergic activity.

In an embodiment, the anti-allergic activity may include inhibiting degranulation in activated mast cells. In detail, the anti-allergic activity may include inhibiting secretion of β-hexosaminidase, histamine, prostaglandins, and serine proteases from activated mast cells.

In an embodiment, the anti-allergic activity may include reducing an IgE concentration in serum or reducing binding of IgE to a surface receptor of mast cells. In addition, the anti-allergic activity may include inhibiting MCPT-1 levels in serum.

In an embodiment, the allergic disease may include an allergic reaction caused by house dust, fungi, mites, or hair, skin or excrement of an animal.

The allergic disease may be selected from the group consisting of edema, anaphylaxis, rhinitis, allergic rhinitis, asthma, allergic conjunctivitis, allergic dermatitis, allergic tympanitis, atopic dermatitis, contact dermatitis, urticaria, chronic spontaneous urticaria, hives, dry eye, pruritus, anaphylactic shock, pollen allergy, bacterial allergy, fungal allergy, viral allergy, insect allergy, food allergy, drug allergy, and respiratory allergy.

The pharmaceutical composition may additionally include a pharmaceutically acceptable diluent or carrier. The diluent may be lactose, corn starch, soybean oil, microcrystalline cellulose, or mannitol, and a glidant may be magnesium stearate, talc, or a combination thereof. The carrier may be an excipient, a disintegrant, a binder, a glidant, or a combination thereof. The excipient may be microcrystalline cellulose, lactose, low-substituted hydroxy cellulose, or a combination thereof. The disintegrant may be calcium carboxymethylcellulose, sodium starch glycolate, calcium dihydrogen phosphate, or a combination thereof. The binder may be polyvinylpyrrolidone, low-substituted hydroxypropyl cellulose, hydroxypropyl cellulose, or a combination thereof. The glidant may be magnesium stearate, silicon dioxide, talc, or a combination thereof.

The pharmaceutical composition may be formulated as an oral dosage form or a parenteral dosage form. The oral dosage form may be a granule, a powder, a liquid, a tablet, a capsule, a dry syrup, or a combination thereof. The parenteral dosage form may be an injection.

Another aspect provides a cosmetic composition for preventing or ameliorating allergic diseases, the cosmetic composition including, as an active ingredient, a Lactiplantibacillus plantarum strain, a lysate derived from the strain, a culture derived from the strain, or a mixture thereof.

The terms “strain,” “anti-allergic activity,” and “allergic disease” are as defined above.

In an embodiment, the allergic disease may be a skin allergic disease.

In an embodiment, the cosmetic composition may have anti-allergic activity.

The cosmetic composition may be, for example, a softening toner, a nourishing toner, a massage cream, a nourishing cream, an essence, a pack, a gel, an ampoule, or a skin-adhesive cosmetic formulation.

The ingredients included in the cosmetic composition may include, in addition to the composition, ingredients commonly used in the cosmetic composition as an active ingredient. For example, common auxiliary agents, such as an antioxidant, a stabilizer, a solubilizer, a vitamin, a pigment, and a flavor, and carriers may be included.

Another aspect provides a feed composition for preventing or ameliorating allergic diseases, the feed composition including, as an active ingredient, a Lactiplantibacillus plantarum strain, a lysate derived from the strain, a culture derived from the strain, or a mixture thereof.

In an embodiment, the feed composition may have anti-allergic activity.

The terms “strain,” “anti-allergic activity,” and “allergic disease” are as defined above.

The “feed” may refer to any natural or artificial formulated feed, one-meal feed, or ingredients of the one-meal feed that an animal eats, ingests, and digests or that is suitable for these. Types of the feed are not particularly limited, and any feed commonly used in the art may be used. Non-limiting examples of the feed may include: vegetable feeds such as grains, root fruits, food processing by-products, algae, fibers, pharmaceutical by-products, fat and oils, starches, gourds or grain by-products; and animal feeds such as proteins, fat-free substances, fat and oils, minerals, unicellular proteins, zooplanktons, or food.

The feed composition may be prepared by adding the mixed strain composition in an appropriate effective concentration range according to various feed preparation methods known in the art, and may be used as a feed additive composition for the purpose of preventing or ameliorating allergic diseases.

When the composition is prepared as a feed additive, the composition may be prepared in the form of a 20 to 90% concentrated solution or in the form of a powder or granule. The feed additive may additionally include any one or at least one of organic acids, such as citric acid, fumaric acid, adipic acid, lactic acid, and malic acid, phosphates, such as sodium phosphate, potassium phosphate, acid pyrophosphate, and polyphosphate (polyphosphate), and natural antioxidants, such as polyphenol, catechin, alpha-tocopherol, rosemary extract, vitamin C, green tea extract, licorice extract, chitosan, tannic acid, and phytic acid. When prepared as a feed, the composition may be formulated in the form of a conventional feed, and may include conventional feed ingredients.

The feed additive may be administered to an animal alone or in combination with other feed additives in an edible carrier. In addition, the feed additive may be easily administered to an animal as a topdressing, or by mixing the feed additive directly into animal feed, or as an oral formulation separate from the feed. When administered separately from animal feed, the feed additive may be formulated into an immediate-release formulation or a sustained-release formulation in combination with a pharmaceutically acceptable edible carrier, as is well known in the art. These edible carriers may be solid or liquid, for example, corn starch, lactose, sucrose, soybean flakes, peanut oil, olive oil, sesame oil, and propylene glycol. When a solid carrier is used, the feed additive may be a tablet, a capsule, a powder, a troche or lozenge, or topdressing in a non-dispersible form. When a liquid carrier is used, the feed additive may be formulated as a gelatin soft capsule, or a syrup or suspension, an emulsion, or a solution. In addition, the feed and feed additive may include an auxiliary agent, such as a preservative, a stabilizer, a wetting agent or emulsifying agent, a solution accelerator, and the like. The feed additive may be used by adding it to animal feed by immersion, spraying, or mixing.

The feed or feed additive may be applied to diets of many animals including mammals, poultry, and fish. The mammals include pigs, cows, horses, sheep, rabbits, goats, rodents, and experimental rodents such as rats, hamsters, and guinea pigs, as well as companion animals (e.g., dogs and cats), the poultry may include chickens, turkeys, ducks, geese, pheasants, and quail, and the fish may include carp, crucian carp, and trout, but examples are not limited thereto.

Another aspect provides a method of preventing, ameliorating, or treating a condition of a subject, the method including treating or administering an effective amount of the composition to a subject in need thereof.

In an embodiment, the condition of the subject may be an immunoglobulin E (IgE)-mediated disease or pathologic condition or an allergic disease-related condition.

The administration may be by any method known in the art. The administration may include direct administration to a subject by any means, for example, by a route such as by directly into the subject by any means, for example, intravenous, intramuscular, oral, transdermal, mucosal, intranasal, intratracheal or subcutaneous administration. The administration may be systemically administered or locally administered.

The subject may be a mammal, for example, a human, a cow, a horse, a pig, a dog, a sheep, a goat, or a cat. The subject may be a subject in need of ameliorating a condition related to inflammation or a condition related to a bacterial infection.

The administration may include administration of the composition according to an embodiment at a dose of 0.00001 mg to 1,000 mg per subject per day, for example, 0.00001 mg to 500 mg, 0.00001 mg to 100 mg, 0.00001 mg to 50 mg, 0.00001 mg to 25 mg, 1 mg to 1,000 mg, 1 mg to 500 mg, 1 mg to 100 mg, 1 mg to 50 mg, 1 mg to 25 mg, 5 mg to 1,000 mg, 5 mg to 500 mg, 5 mg to 100 mg, 5 mg to 50 mg, 5 mg to 25 mg, 10 mg to 1,000 mg, 10 mg to 500 mg, 10 mg to 100 mg, 10 mg to 50 mg, or 10 mg to 25 mg, per subject per day. However, the dosage may be prescribed in various ways depending on factors, such as a formulation method, an administration method, the age, weight, gender, pathological condition, diet, administration time, administration route, excretion rate, and response sensitivity of a patient, and a person skilled in the art may appropriately adjust the dosage by considering these factors. The number of administrations may be once daily, or at least twice within the range of clinically acceptable side effects, the number of administration sites may be one or at least two, and the total number of days of administration may be from 1 to 30 days at intervals of 2 to 5 days per treatment. If necessary, the same treatment may be repeated after an appropriate period of time. For non-human animals, the same dosage as that for humans per kg may be administered, or for example, the dosage is calculated based on the volume ratio (e.g., average value) of organs (e.g., the heart) between a target animal and humans and then administered.

### Advantageous Effects of Disclosure

The strain according to an aspect, the strain-derived lysate and culture, the extract of the culture, or a mixture thereof have effects that can be useful in the prevention, amelioration, and treatment of allergic diseases.

### BRIEF DESCRIPTION OF DRAWINGS

FIG. 1 is a graph showing an effect of inhibiting degranulation of RBL-2H3 mast cells by a Lactiplantibacillus plantarum HD-02 culture supernatant.

FIG. 2 is a graph showing an effect of inhibiting an allergic reaction by a Lactiplantibacillus plantarum HD-02 strain in a mouse model with anti-DNP IgE-induced passive cutaneous anaphylaxis.

FIG. 3 is a graph showing an effect of inhibiting ear edema by a Lactiplantibacillus plantarum HD-02 strain in a mouse model with MC903-induced atopic dermatitis.

FIG. 4 is a graph showing an effect of inhibiting IgE reduction in serum by a Lactiplantibacillus plantarum HD-02 strain in a mouse model with MC903-induced atopic dermatitis.

FIG. 5 is a graph showing an effect of inhibiting MCPT-1 reduction in serum by a Lactiplantibacillus plantarum HD-02 strain in a mouse model with MC903-induced atopic dermatitis.

FIG. 6 is a graph showing an effect of alleviating an atopic dermatitis symptom by a Lactiplantibacillus plantarum HD-02 strain in a mouse model with house dust mite-induced atopic dermatitis.

FIG. 7 is a graph showing an effect of reducing IgE in serum by a Lactiplantibacillus plantarum HD-02 strain in a mouse model with house dust mite-induced atopic dermatitis.

FIG. 8 is a graph showing an effect of reducing transepidermal water loss in serum by a Lactiplantibacillus plantarum HD-02 strain in a mouse model with house dust mite-induced atopic dermatitis.

FIG. 9 is a graph showing an effect of reducing itching by a Lactiplantibacillus plantarum HD-02 strain in a mouse model with house dust mite-induced atopic dermatitis.

FIG. 10 is a graph showing an effect of reducing the number of B cells and T cells in axillary lymph nodes and the total number of cells by a Lactiplantibacillus plantarum HD-02 strain in a mouse model with house dust mite-induced atopic dermatitis.

FIG. 11 is a graph showing an effect of reducing Th1 cells and Th2 cells in axillary lymph nodes by a Lactiplantibacillus plantarum HD-02 strain in a mouse model with house dust mite-induced atopic dermatitis.

FIG. 12 is a graph showing an effect of reducing skin thickness by a Lactiplantibacillus plantarum HD-02 strain in a mouse model with house dust mite-induced atopic dermatitis.

FIG. 13 is a graph showing an effect of inhibiting infiltration and degranulation of mast cells by a Lactiplantibacillus plantarum HD-02 strain in a mouse model with MC903-induced atopic dermatitis.

### MODE FOR THE INVENTION

Hereinafter, preferable Examples are presented to help understanding of the present disclosure. However, the following examples are only presented for easier understanding of the present disclosure, and the contents of the present disclosure are not limited by the following examples. Examples can undergo various modifications, and thus examples are not limited to those disclosed below and can be implemented in various forms.

### Example 1. Isolation and Identification of Lactiplantibacillus plantarum Strains

### 1.1. Isolation of Strains

To isolate Lactiplantibacillus plantarum strains, a variety of kimchi obtained at home were used. A 25 g the kimchi sample was diluted in 225 ml of a 0.85% NaCl solution, placed in a wire filter sample bag for a homogenizer, and homogenized for 5 minutes using a stomacher. The filtrate was subjected to decimal dilution, spread on an MRS plate medium (pH 5.5, Difco, USA), and cultured anaerobically at 37° C. for 48 hours. After the culturing, single colonies were distinguished according to morphology and color, and subjected to pure isolation through subculturing three times. Then, a strain that was positive and negative in Gram staining and catalase test, respectively, was selected and arbitrarily named HD-02, and was finally selected as a Lactiplantibacillus plantarum HD-02 strain (hereinafter referred to as ‘HD-02’).

### 1.2. Molecular Biological Identification of Selected Strain

To conduct the 16S rRNA sequencing of the HD-02 strain, the 16S rRNA genes were amplified using bacterial universal primers, and then subjected to sequencing (Macrogen Inc, Korea). PCR was performed using the primer sequences listed in Table 1, and the analysis results are as shown in SEQ ID NO: 1.

TABLE 1

| Gene | Primer | Sequence (5′→3′) |
|---|---|---|
| 16S rRNA | 27F | AGAGTTTGATCMTGGCTCAG (SEQ ID NO: 3) |
| | 1492R | TACGGYTACCTTGTTACGACTT (SEQ ID NO: 4) |

As a result of interpreting the 16S rRNA base sequence of each strain by using the sequence comparison program (BLAST) of the National Center for Biotechnology Information (NCBI) in the United States, it was confirmed that the HD-02 strain had 99% or more identity in the 16S rRNA gene with several strains belonging to the species of Lactiplantibacillus plantarum including a standard strain of Lactiplantibacillus plantarum (JCM 1149) (Table 2).

TABLE 2

| | | | Query | | |
|---|---|---|---|---|---|
| | Max | Total | coverage | Identity | |
| Description | Score | score | (%) | (%) | Accession |
| Lactiplantibacillus pentosus strain 124- | 2763 | 2763 | 98 | 99.87 | NR_029133.1 |
| 2 16S ribosomal RNA, partial sequence | | | | | |
| Lactiplantibacillus paraplantarum strain | 2758 | 2758 | 98 | 99.87 | NR_025447.1 |
| DSM 10667 16S ribosomal RNA, partial | | | | | |
| sequence | | | | | |
| Lactiplantibacillus plantarum strain | 2754 | 2754 | 98 | 99.73 | NR_115605.1 |
| JCM 1149 16S ribosomal RNA, partial | | | | | |
| sequence | | | | | |
| Lactiplantibacillus plantarum strain CIP | 2750 | 2750 | 98 | 99.87 | NR_104573.1 |
| 103151 16S ribosomal RNA, partial | | | | | |
| sequence | | | | | |

The Lactiplantibacillus plantarum has at least 99% 16S rRNA sequence identity with closely related species such as Lactiplantibacillus pentosus, and thus, for more accurate identification, analysis was performed on the recA gene which is one of taxonomic biomarkers other than the 16S rRNA gene. The gene was amplified using the recA-specific primers shown in Table 3, and then sequenced (Bionics Co., Ltd, Korea). The analysis results are as shown in SEQ ID NO: 2.

TABLE 3

| Gene | Primer | Sequence (5′→3′) |
|---|---|---|
| recA | L. p_recA_F | CTGATGCACGGAAAGCAGC (SEQ ID NO: 5) |
| | L. p_recA_R | CGCTTAAATGGYGGYGCMACC (SEQ ID NO: 6) |

As a result of interpreting the recA gene sequences of each strain by using the sequence comparison program (BLAST) of the NCBI in the United States, it was confirmed that the HD-02 strain had at least 99% identity to the recA gene of the Lactiplantibacillus plantarum (Table 4).

TABLE 4

| | | | Query | | |
|---|---|---|---|---|---|
| | Max | Total | coverage | Identity | |
| DESCRIPTION | Score | Score | (%) | (%) | Accession |
| Lactiplantibacillus | 1301 | 1301 | 100 | 99.72 | CP053912.1 |
| plantarum subsp. | | | | | |
| plantarum strain G1 | | | | | |
| chromosome. | | | | | |
| Complete genome | | | | | |
| Lactiplantibacillus | 1301 | 1301 | 100 | 99.72 | CP053571.1 |
| plantarum strain | | | | | |
| CNEI-KCA4 | | | | | |
| chromosome. | | | | | |
| Complete genome | | | | | |
| Lactiplantibacillus | 1301 | 1301 | 100 | 99.72 | CP052869.1 |
| plantarum strain | | | | | |
| AMT74419 | | | | | |
| chromosome. | | | | | |
| Complete genome | | | | | |
| Lactiplantibacillus | 1301 | 1301 | 100 | 99.72 | CP050805.1 |
| plantarum strain SPC- | | | | | |
| SNU 72-2 | | | | | |
| chromosome. | | | | | |
| Complete genome | | | | | |

### 1.3. Morphological and Physiological Characterization of Selected Strains

Morphological and physiological characteristics of the HD-02 strain were determined according to the contents of Bergy's manual of systematic bacteriology and using the API50CHL kit (Biomerieux SA, France). The morphological and physiological characteristics of the HD-02 strain are summarized in Table 5, and the sugar fermentation characteristics of the strain are summarized in Table 6.

TABLE 5

| | |
|---|---|
| Strain name | HD-02 |

| | | |
|---|---|---|
| Shape of | Shape of cells | Rod-type |
| bacteria | Mobility | None |
| | Gram staining | Positive |
| Shape of | Color | Ivory white |
| colony | Transparency | Opaque |
| | shape | Circular |
| | Surface | Smooth |
| Physiological | Catalase | Negative |
| properties | Effect on oxygen | Facultative |
| | | anaerobic |

TABLE 6

| Type of sugar | HD-02 |
|---|---|
| Glycerol | − |
| Erythritol | − |
| D-arabinose | − |
| L-arabinose | + |
| D-ribose | + |
| D-xylose (DXYL) | − |
| L-xylose | − |
| D-xylose (ADO) | − |
| Methyl-beta-D-xylopyranoside | − |
| D-glactose | + |
| D-glucose | + |
| D-fructose | + |
| D-mannose | + |
| L-sorbose | − |
| L-rhamnose | − |
| Dulcitol | − |
| Inositol | − |
| D-mannitol | + |
| D-sorbitol | + |
| Methyl-alpha-D-mannopyranoside | − |
| Methyl-alpha-D-glucopyranoside | − |
| N-acetylglucosamine | + |
| Amygdalin | + |
| Arbutin | + |
| Esculin ferrous citrate | − |
| Salicin | + |
| D-cellobiose | + |
| D-maltose | + |
| D-lactose (bovine origin) | − |
| D-mellibiose | + |
| D-sucrose | + |
| D-trehalose | + |
| Inulin | − |
| D-melegitoz | + |
| D-raffinose | − |
| Amidon (starch) | − |
| Glycogen | − |
| Xylitol | − |
| Gentiobiose | + |
| D-turanose | + |
| D-rixous | − |
| D-tagatose | − |
| D-fucose | − |
| L-fucose | − |
| D-arabitol | − |
| L-arabitol | − |
| Potassium gluconate | − |
| Potassium 2-ketogluconate | − |
| Potassium 5-ketogluconate | − |

Positive: (+)/Negative: (−). As a result of the fermentation characteristic analysis, the HD-02 strain showed 66.8% similarity to the Lactiplantibacillus plantarum.

Based on the results of the 16S rRNA base sequencing and the recA gene sequencing and the morphological and physiological characteristics, the isolated strain was named Lactiplantibacillus plantarum HD-02, and the HD-02 strain was deposited at the Korean Agricultural Culture Collection (KACC) on Aug. 26, 2022 (Accession No.: KACC 81228BP).

### Experimental Example 1. Effect of Inhibiting Degranulation of RBL-2H3 Mast Cells by L. plantarum HD-02 Culture Supernatant

From activated mast cells causing allergic reactions, culture supernatants of various Lactiplantibacillus plantarum strains including the L. plantarum HD-02 strain were each treated, and the inhibitory effect thereof on degranulation was screened by measuring secretion of β-hexosaminidase.

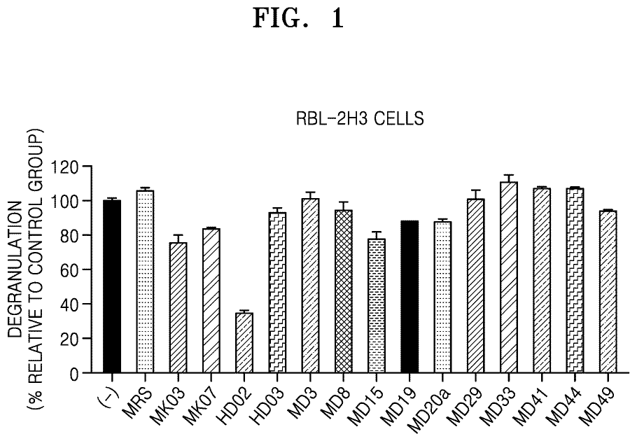

RBL-2H3 cells were suspended in an MEM medium supplemented with 15% FBS in a 24-well plate, seeded at a concentration of 1.5×10⁵ cells per well, and sensitized by culturing with 20 ng/mL DNP-IgE for about 12 hours. After washing the cells once with PIPES buffer (25 mM PIPES, 119 mM NaCl, 5 mM KCl, 0.4 mM MgCl2, 1 mM CaCl2, 5.6 mM glucose, 0.1% BSA), the culture supernatant of various L. plantarum strains was diluted with PIPES buffer at 1:15 and pretreated in each well for 2 hours. To obtain the culture supernatant, the L. plantarum strain was cultured in an MRS medium and precipitated by centrifugation, and only the supernatant was collected and then filtered through a 0.22 μm filter. Afterwards, 25 ng/mL DNP-HSA (antigen) was added to the supernatant and caused a stimulation for 15 minutes, and the reaction was stopped by cooling on ice for 5 minutes. The supernatant was transferred to an e-tube, washed once with PIPES buffer, and the cells remaining on the plate were disrupted with 0.1% Triton X-100. 30 μL of each of the supernatant and the lysed cell sample was mixed with a substrate buffer (1 mM 4-Nitrophenyl N-acetyl-3-D-glucosaminide, 0.1 M sodium citrate) in a 96-well plate and reacted at 37° C. for 1 hour. After the reaction was terminated by adding 0.1 M carbonate buffer, the absorbance was measured at a wavelength of 405 nm using a microplate reader, and the results are shown in FIG. 1.

As shown in FIG. 1, 14 different supernatants of the L. plantarum strain were screened, and as a result, it was confirmed that the culture supernatant of the L. plantarum HD-02 strain had the greatest effect of inhibiting degranulation of mast cells.

### Experimental Example 2. Conformation of Inhibitory Effect on IgE-Mediated Allergic Reaction in L. plantarum HD-02 Strain-Administered Group in Animal Model with Passive Cutaneous Anaphylaxis

In an animal model with passive cutaneous anaphylaxis (PCA) induced by anti-DNP IgE, the inhibitory effect on allergic reaction in the L. plantarum HD-02 strain was confirmed.

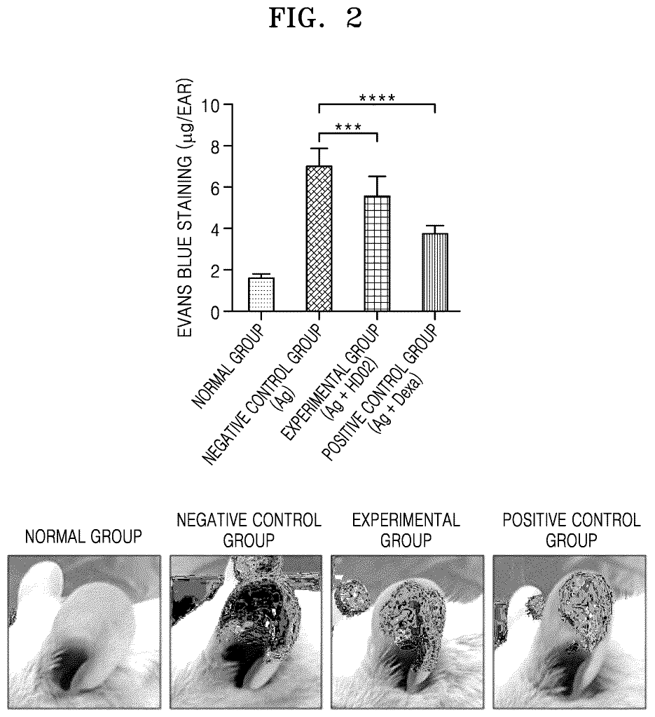

Four-week-old BALB/c mice were obtained and acclimatized for one week, and the lyophilized L. plantarum HD-02 strain was suspended in D-PBS and administered orally to each mouse at a dose of 1×10¹⁰ CFU daily from 2 days before IgE sensitization until immediately before the end of the test. As a positive control group, dexamethasone (Dexa) was used at a concentration of 10 mg/kg. To induce PCA allergic reaction, 20 ng of anti-DNP IgE was injected locally into one ear of mice. About 24 hours later, DNP-HSA was diluted at a concentration of 0.5 mg/mL in 5 mg/ml of Evans blue staining dye, and then 200 μL thereof was administered into the tail vein. After 30 minutes, the mice were euthanized, the ears were removed, placed in 700 μL of formamide, and the dye exuded into the ear tissue was extracted at 63° C. for 12 hours, and the absorbance of the supernatant was measured at a wavelength of 620 nm. The quantity of the exuded Evans Blue was measured using a standard quantitative curve and is shown in FIG. 2.

As shown in FIG. 2, compared to the negative control group (Ag), the model with PCA allergic reaction caused by anti-DNP IgE showed that the experimental group (Ag+HD-02) administered with the L. plantarum HD-02 strain exhibited the anti-allergic effect by inhibiting allergic reactions by about 20.4%.

### Experimental Example 3. Conformation of Inhibitory Effect on Allergic Reactions by L. plantarum HD-02 Stain-Administered Group in Animal Model with MC903-Induced Atopic Dermatitis

3.1. Inhibitory Effect on Ear Edema by L. plantarum HD-02 Strain-Administered Group

In an animal model with MC903-induced atopic dermatitis, the inhibitory effect on ear edema by the L. plantarum HD-02 strain was confirmed.

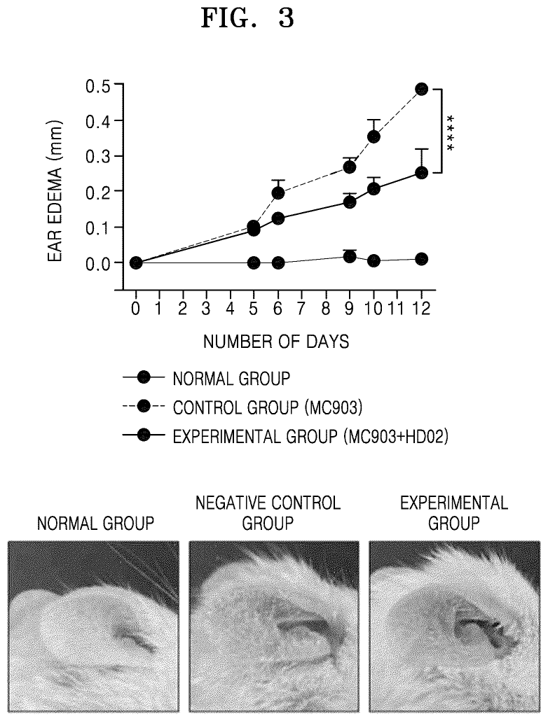

Six-week-old BALB/c mice were obtained and acclimatized for one week, and the lyophilized L. plantarum HD-02 strain was suspended in D-PBS and administered orally to each mouse at a dose of 2×10⁹ CFU daily from 7 days before inducing atopic dermatitis until immediately before the end of the test. After correcting the mice to induce atopic dermatitis, 20 μL of 2 nmol MC903 (Calcipotriol, C27H4003) dissolved in ethanol was applied to both ears of the mice in the disease-induced group every day for 12 days, while only ethanol was applied to the normal group. To determine the degree of ear edema caused by atopic dermatitis, the thickness of the mouse ears was measured daily at the same time, and is shown in FIG. 3. Here, changes in the ear edema were measured using the value obtained by subtracting the ear thickness measured before disease induction from the ear thickness measured daily after disease induction.

As shown in FIG. 3 with respect to the model with the MC903-induced atopic dermatitis, it was confirmed that, compared to the control group (MC903), the experimental group (MC903+HD-02) administered with the L. plantarum HD-02 strain exhibited inhibitory effect of ear edema by about 48.4%.

3.2. Inhibitory Effect on Reduction of Total IgE in Serum by L. plantarum HD-02 Strain-Administered Group

IgE overexpression is the most representative marker of atopic dermatitis. To confirm the total IgE concentration in serum from the MC903-induced atopic dermatitis animal model of Experimental Example 3.1, a mouse IgE ELISA Kit (Biolegend #432404) was used for the measurement.

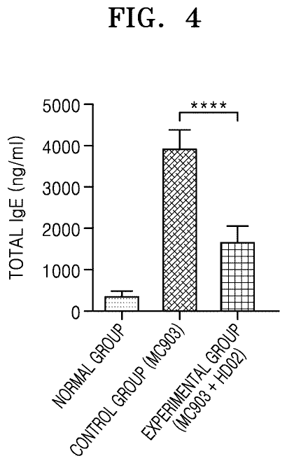

Microwell plates were coated with capturing antibodies diluted in coating buffer and caused a reaction overnight at 4° C. The cells were then washed four times with washing buffer and blocked with an assay diluent for 1 hour at room temperature. Following four times of a washing process, a standard sample and a serum sample diluted at 1:200 were added and reacted at room temperature for 2 hours. Following four times of a washing process, detection antibodies were added and caused a reaction at room temperature for 1 hour. Following four times of a washing process, avidin-HRP was added and caused a reaction at room temperature for 30 minutes. Following five times of a washing process, a TMB substrate solution was added and caused a reaction at room temperature for 20 minutes while blocking light. A stop solution was added to each well to stop the color reaction, and the absorbance was measured at 450 nm using an ELISA reader. The results are shown in FIG. 4.

As shown in FIG. 4, it was confirmed that the total IgE concentration in serum rapidly increased in the control group (MC903) compared to the normal group, and that the experimental group (MC903+HD-02) administered with the L. plantarum HD-02 strain exhibited an effect of reducing the total IgE concentration by about 57.2%.

3.3. Effect on Reduction of MCPT-1 in Serum by L. plantarum HD-02 Strain-Administered Group

To confirm the concentration of mast cell protease-1 (MCPT-1) in serum from the MC903-induced atopic dermatitis animal model of Experimental Example 3.1, the concentration was measured using a mouse MCPT-1 (mMCP-1) ELISA Kit (Invitrogen #88-7503).

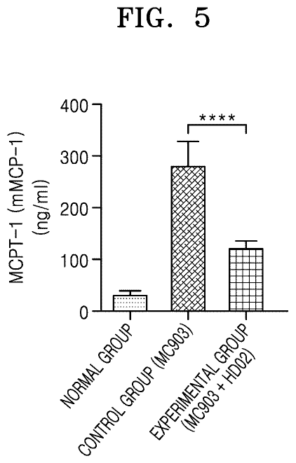

Microwell plates were coated with capturing antibodies diluted in coating buffer and caused a reaction overnight at 4° C. The cells were then washed with a washing buffer three times and blocked with an ELISA/ELISPOT diluent for 1 hour at room temperature. Following three times of a washing process, a standard sample and a serum sample diluted at 1:200 were added and caused a reaction at room temperature for 2 hours. Following three times of a washing process, detection antibodies were added and caused a reaction at room temperature for 1 hour. Following three times of a washing process, avidin-HRP was added and caused a reaction at room temperature for 30 minutes. Following five times of a washing process, a TMB substrate solution was added and caused a reaction at room temperature for 15 minutes while blocking light. A stop solution was added to each well to stop the color reaction, and the absorbance was measured at 450 nm using an ELISA reader. The results are shown in FIG. 5.

As shown in FIG. 5, it was confirmed that the MCPT-1 concentration in serum rapidly increased in the control group (MC903) compared to the normal group, and that the experimental group (MC903+HD-02) administered with the L. plantarum HD-02 strain exhibited an effect of reducing the MCPT-1 concentration in serum by about 56.8%.

### Experimental Example 4. Inhibitory Effect on Allergic Reactions by L. plantarum HD-02 Strain-Administered Group in Animal Model with House Dust-Induced Atopic Dermatitis

4.1. Effects of Alleviating Atopic Dermatitis Symptoms by L. plantarum HD-02 Strain-Administered Group

Inhibitory effects on atopic dermatitis symptoms by the L. plantarum HD-02 strain-administered group in an animal model with house dust-induced atopic dermatitis were confirmed.

Six-week-old NC/Nga mice were obtained and acclimatized for one week, and the hair on the back and upper part of the ears of the mice was cleanly removed. To remove fat components of skin and disrupt the cuticle barrier, 150 μL of 4% SDS aqueous solution was sprayed onto the hair-removed area, and the sprayed area was completely dried and evenly coated with 100 mg of Biostar AD (natural mite-derived ingredient, Dermatophagoides farinae) ointment. An inducing substance was applied twice a week for three weeks to induce atopic dermatitis, and then applied once a week for the next eight weeks to maintain atopic dermatitis. Administration of the L. plantarum HD-02 strain began at the completion point of the 3-week atopic dermatitis induction period, and was done orally at a dose of 2×10⁹ CFU per mouse until shortly before the end of the experiment. A positive control group using dexamethasone was applied onto the dorsal skin twice weekly at a concentration of 0.1%, i.e., 100 μL per subject.

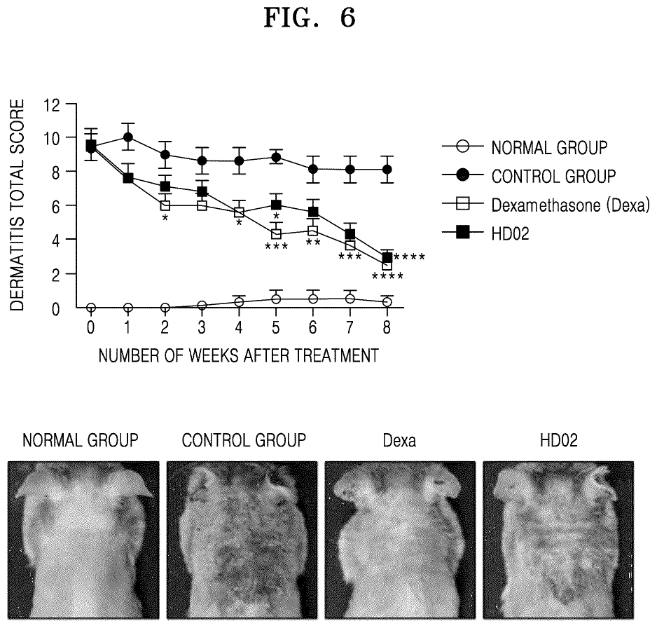

Evaluation of severity of atopic dermatitis symptoms was performed weekly, and expressed as the sum of scores (Dermatitis scores) for each of the following four items total score. The evaluation items were erythema/hemorrhage, scarring/dryness, edema, and excoriation/erosion, and the scores of 0 (no symptoms), 1 (weak), 2 (moderate), and 3 (severe) for each of the four items were judged and summed, and the results are shown in FIG. 6.

As shown in FIG. 6, the severity of atopic dermatitis score of the control group (house dust mite-induced) was measured as an average of 8.17 points, while the score of the positive control group (Dexa) was measured as 2.50, showing an effect of alleviating atopic dermatitis symptoms of about 69.4% compared to the control group. In addition, it was confirmed that the severity of atopic dermatitis score of the experimental group (house dust mite-induced+HD-02) was measured to as an average of 3.00, showing an effect of alleviating atopic dermatitis symptoms of about 63.3%, which was similar to that of the positive control group (Dexa).

4.2. Effect of Reducing Total IgE in Serum by L. plantarum HD-02 Strain-Administered Group

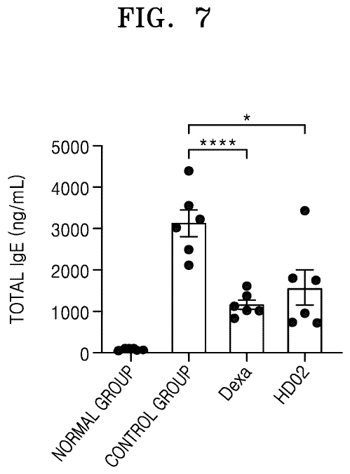

To determine the total IgE concentration in serum from the model with house dust mite-induced atopic dermatitis (severe atopic dermatitis model) of Experimental Example 4.1, the concentration was measured using a Mouse IgE ELISA Kit in the same manner as in Experimental Example 3.2, and the results are shown in FIG. 7.

As shown in FIG. 7, the total IgE concentration in serum rapidly increased in the control group (house dust mite-induced) compared to the normal group (no house dust mite-induced). Compared to the control group (house dust mite-induced), the total IgE inhibition in serum was about 63.7% in the positive control group (Dexa) and 50.6% in the experimental group (house dust mite-induced+HD-02).

4.3. Effects of Reducing Transepidermal Moisture Evaporation by L. plantarum HD-02 Strain-Administered Group

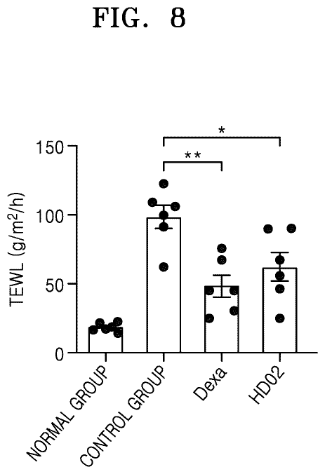

To determine the transepidermal moisture loss caused by impaired skin barrier function from the model with house dust mite-induced atopic dermatitis (severe atopic dermatitis model) of Experimental Example 4.1, the moisture evaporation rate from the skin surface was measured using a Tewameter TM Nano device, and the results are shown in FIG. 8.

As shown in FIG. 8, the control group (house dust mite-induced) showed a rapid increase in the transepidermal water loss compared to the normal group (no house dust mite-induced). Compared to the control group, the reduction in transepidermal water loss values was about 51.3% in the positive control group (Dexa) and was about 36.9% in the experimental group (house dust mite-induced+HD-02). Accordingly, the effect of ameliorating the skin barrier by the L. plantarum HD-02 was confirmed.

4.4. Effects of Reducing Itching by L. plantarum HD-02 Strain-Administered Group

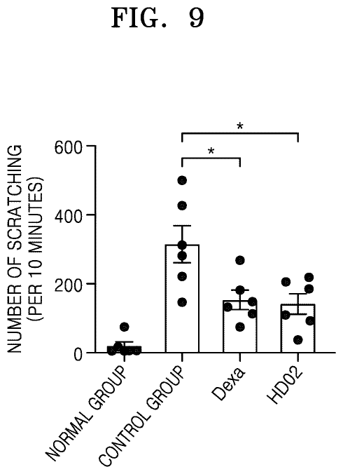

From the model with house dust mite-induced atopic dermatitis (severe atopic dermatitis model) of Experimental Example 4.1, itchy sense demonstrating the degree of itchiness in atopic skin lesions was confirmed as an important evaluation index of the disease model. Mice in all groups were filmed for 10 minutes for each mouse, and the scratching behavior frequency of the filmed mice was measured, and the results are shown in FIG. 9.

As shown in FIG. 9, it was confirmed that the control group (house dust mite-induced) increased the number of scratching by about 18.7 times compared to the normal group (no house dust mite-induced), and that the itching relief effect was about 51.5% in the positive control group (Dexa) and about 55.2% in the experimental group (house dust mite-induced+HD-02) compared to the control group.

4.5. Confirmation of Changes in Cell Composition in Axillary Lymph Node by L. plantarum HD-02 Strain-Administered Group

From the model with house dust mite-induced atopic dermatitis (severe atopic dermatitis model) of Experimental Example 4.1, changes in the composition of immune cells in the axillary lymph node that is an important immune tissue of a disease model were confirmed.

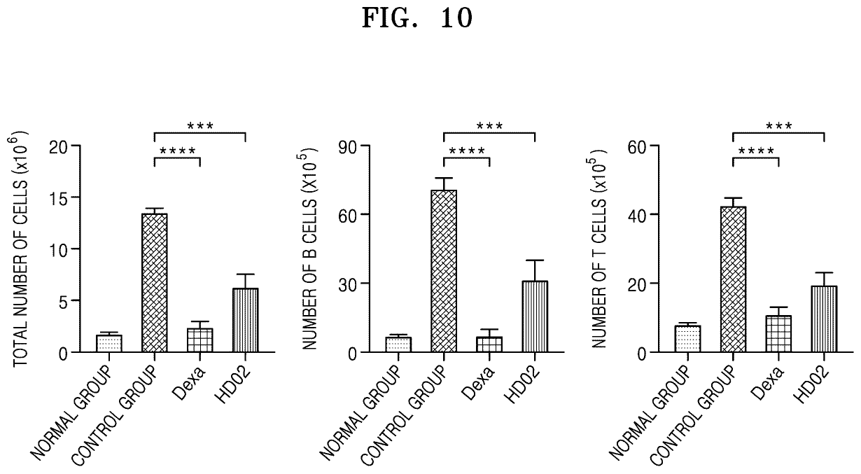

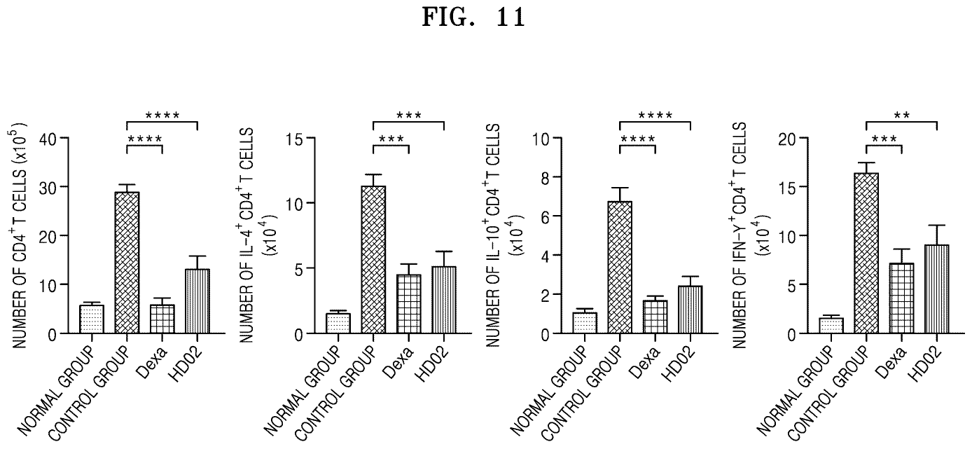

The axillary lymph node of each mouse was extracted and a single cell suspension was obtained. Then, the cells were counted and dispensed into a 96-well U-bottom microwell plate at a cell count of 1.0×10⁶ cells per well. After 4 hours of stimulation with eBioscience™ Cell Stimulation Cocktail (plus protein transport inhibitors), the cells were stained with anti-CD3-Pacific blue, anti-CD4-PE-Cy7, anti-IL-10-FITC, anti-IL-4-PE, and anti-IFN-γ-APC antibodies and subjected to FACS analysis. The results are shown in FIGS. 10 and 11.

As shown in FIG. 10, it was confirmed that the total number of cells in the axillary lymph node was increased by about 7.5 times in the control group (house dust mite-induced) that induced atopic dermatitis compared to the normal group (in which no house dust mite was induced), and that the number of axillary lymph node cells was decreased by about 81.7% in the positive control group (Dexa) and about 53.7% in the experimental group (house dust mite-induced+HD-02) compared to the control group. In addition, as shown in FIG. 11, it was confirmed that B cells, T cells, and Th1 and Th2 cells in the axillary lymph node also showed a significant decrease by administration of the L. plantarum HD-02 compared to the control group.

4.6. Confirmation of Changes in Skin Thickness and Histological Changes by L. plantarum HD-02 Strain-Administered Group

From the model with house dust mite-induced atopic dermatitis (severe atopic dermatitis model) of Experimental Example 4.1, the efficacy of the L. plantarum HD-02 strain on skin thickness increase and mast cell infiltration in response to inflammation was determined.

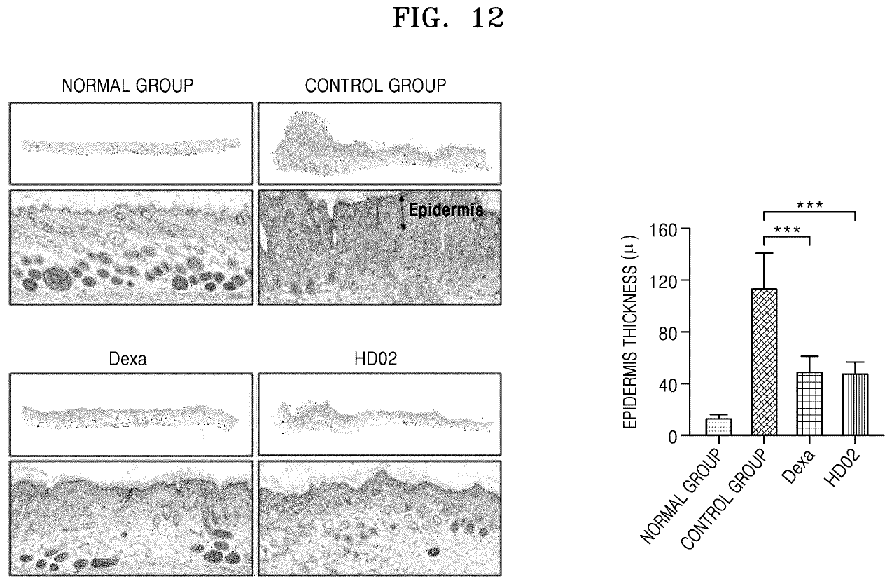

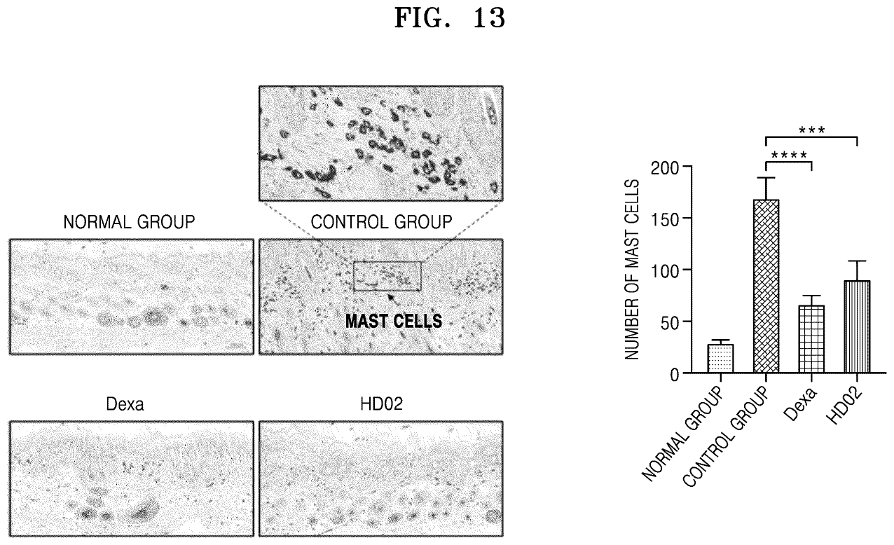

After the dorsal skin of the mouse was extracted, the tissue was fixed with 10% formalin, and paraffin blocks were made sectioned at a thickness of 4 μm to prepare a slide. Hematoxylin&Eosin (H&E) staining was used to observe accumulation of lymphocytes in the skin tissue and changes in epidermal thickness, and toluidine blue staining was used to measure the number of mast cells to determine the degree of tissue infiltration. The results are shown in FIGS. 12 and 13.

As shown in FIG. 12, it was confirmed that the thickness of the epidermal layer increased by about 8.8 times in the control group (house dust mite-induced) that induced atopic dermatitis compared to the normal group (no house dust mite-induced), and that the thickness of the epidermal layer was inhibited by about 56.7% in the positive control group (Dexa) and about 57.3% in the experimental group (house dust mite-induced+HD-02) compared to the control group. In addition, it was confirmed that the degree of accumulation of lymphocytes was significantly reduced to the level of the positive control group (Dexa) in the experimental group compared to the control group.

Furthermore, as shown in FIG. 13, infiltration of mast cells in inflamed tissue was confirmed, and it was confirmed that the number of mast cells was increased by about 6.2 times in the control group that induced atopic dermatitis compared to the normal group, and that, compared to the control group, the number of mast cells was significantly reduced by about 61.4% in the positive control group (Dexa) and about 47.0% in the experimental group.

The results above indicate that the L. plantarum HD-02 strain may be useful for the prevention, amelioration, or treatment of atopic dermatitis or allergic diseases.

The foregoing descriptions are only for illustrating the present disclosure, and it will be apparent to a person having ordinary skill in the art to which the present invention pertains that the embodiments disclosed herein can be easily modified into other specific forms without changing the technical spirit or essential features. Therefore, it should be understood that Examples described herein are illustrative in all respects and are not limited.

1. A method of preventing or treating an allergic disease, the method comprising administering an effective amount of a composition to a subject in need thereof, the composition comprising a Lactiplantibacillus plantarum HD-02 strain deposited with Accession No. KACC81228BP, a lysate of the strain, a culture of the strain, or a mixture thereof.

2. The method of claim 1, wherein the composition inhibits production of a helper T cell type 2 (Th2)-mediated cytokine.

3. The method of claim 1, wherein the composition reduces a concentration of IgE in serum or reduces binding of IgE to a surface receptor of a mast cell.

4. The method of claim 1, wherein the composition further comprises at least one additive selected from the group consisting of a preservative, a dye, an emulsifier, a sweetener, a stabilizer, a flavor enhancer, a flavoring agent, and an acidifier.

5. The method of claim 1, wherein the composition is in a dosage form selected from the group consisting of a solution, an emulsion, a viscous mixture, a powder, a granule, a tablet, and a capsule.

6-8. (canceled)

9. The method of claim 1, wherein the composition has an inhibitory effect on degranulation of a mast cell.

10. The method of claim 1, wherein the composition inhibits i) IgE expression in serum or ii) MCPT-1 levels in serum.

11. The method of claim 1, wherein the allergic disease includes an allergic reaction caused by house dust, fungi, mites, or hair, skin or excrement of an animal.

12. The method of claim 1, wherein the allergic disease is selected from the group consisting of edema, anaphylaxis, rhinitis, allergic rhinitis, asthma, allergic conjunctivitis, allergic dermatitis, allergic tympanitis, atopic dermatitis, contact dermatitis, urticaria, chronic spontaneous urticaria, hives, dry eye, pruritus, anaphylactic shock, pollen allergy, bacterial allergy, fungal allergy, viral allergy, insect allergy, food allergy, drug allergy, and respiratory allergy.

13-14. (canceled)

15. The method of claim 1, wherein the step of treating the allergic disease includes alleviating an immune hypersensitivity reaction or ameliorating a skin condition caused by an immune hypersensitivity reaction.

16-21. (canceled)
